(19) United States
(12) Patent Application Publication
(10) Pub. No.: US 2008/0319034 A1
(43) Pub. Date: Dec. 25, 2008

(54) METHOD FOR PRODUCING PRECIOUS MATERIAL FOR ORNAMENTAL ARTICLES AND PRECIOUS MATERIAL FOR ORNAMENTAL ARTICLES WHICH CAN BE OBTAINED IN PARTICULAR BY MEANS OF THIS METHOD

(75) Inventors: Lorenzo BRUNETTI, Basano del Grappa (IT); Tomaso Neri, Mussolente (IT)

Correspondence Address: BROWDY AND NEIMARK, P.L.L.C.;624 NINTH STREET, NW, SUITE 300, WASHINGTON, DC, 20001-5303, US

(73) Assignees: Lorenzo Brunetti, Vicenza (IT); Laura Cerato, Vicenza (IT)

(21) Appl. No.: 12/101,364

(22) Filed: Apr. 11, 2008

(30) Foreign Application Priority Data
Apr. 13, 2007 (IT) PD2007A000137

(51) Int. Cl. C08K 3/10; C08L 75/04; C08L 67/00; A61P 31/00; B22F 3/20; A61K 31/425; C08L 77/00

(52) U.S. Cl. 514/372; 524/589; 524/599; 524/612; 524/435; 419/67

(57) ABSTRACT

Method for producing a precious material for ornamental articles, which envisages introducing, into the plasticization chamber of an extruder at a temperature of between 120 and 220° C., an amount of a Polymer composition and an amount of a metallic powder formed with one or more precious metals having particles with an average size of less than 0.5 micron in order to achieve thorough mixing between the amount of polymer composition in the liquid state and the amount of metallic powder by means of one or more screws provided with mixing segments; and then an extrusion and cutting step in order to obtain an intermediate product with a precious metal content to be treated during a subsequent forming step aimed at obtaining an ornamental article in the desired form.

### TECHNICAL FIELD

The present invention relates to a method for producing precious material for ornamental articles and to precious material for ornamental articles which can be obtained in particular by means of this method.

The method and the precious material according to the present invention may advantageously be used in the manufacturing industry for the production of precious semi-finished articles.

### BACKGROUND ART

As is known, gold, silver and platinum are precious metals which are widely used in the production of articles of jewelry, among other things because of their malleability and ductility and their aesthetic characteristics.

Generally precious metals are not used in the pure state, but alloyed with other metals, in order to modify their mechanical properties, their aesthetic characteristics and in particular their colour or also only the “carat value” and therefore the cost of the ornamental articles obtained with these metals.

In more detail, as is known, in the gold and silverware sector, the precious metal content of a product is usually indicated by means of an index called the “purity index” which is generally expressed as a fraction of the metal, in thousandths, relative to the total weight of the said product. The said index may also be expressed in carats (K), in this case the fraction of metal relative to the overall weight of the product being expressed in twenty-fourths ( 1/24), instead of thousandths as with the purity index. The platinum alloys which are currently used in the precious metals industry have purity indices which are standardized and equal to 850/1000, 900/1000 or 950/1000, while the purity indices for gold typically are equal to 18 carats (75.0%) for southern Europe; 24 carats ‘Chuk Kam’ (minimum 99.0%) for the Far East (China, Hong Kong, Taiwan); 22 carats (91.6%) for India; 21 carats (87.5%) for the Arab countries; and 8-18 carats (33.3-75.0%) for northern Europe and the USA.

Apart from the constraints imposed by the carat value or the minimum amount of precious metal which must be contained in an ornamental article, during production of the alloys for the gold and silver industry it is necessary to take into account also other factors such as the mechanical properties and “anti-allergy” properties.

The mechanical properties must be such as to allow the use of the alloys in the standard processes and treatments employed in the gold and silver industry (ease of melting, weldability, diamond-machinability, possibility of recovering and reusing the waste product, etc.). It is in fact evident that the alloys must at least not require complex processing techniques such that their use becomes uneconomical.

In most cases the gold and silver articles are worn in direct contact with the skin and therefore must ensure anti-allergic properties which allow the said articles to be worn also by persons who are more sensitive.

For this purpose it is known, for example, that the alloys for gold and silverware must ensure a slow release of allergy-producing agents such as nickel and cobalt.

For each precious metal alloy the choice of the purity index and the metals which form the alloy together with the precious metal, in terms of both alloy base elements and alloying elements, therefore generally depends on commercial factors and the type of product which is to be obtained.

The metal elements which are currently most used for gold alloys are: silver (Ag), copper (Cu); zinc (Zn); indium (In); palladium (Pd); nickel (Ni); while those most used for platinum alloys are: cobalt (Co), iridium (Ir), gold (Au), palladium (Pd), rhodium (Rh) and ruthenium (Ru)

In order to satisfy the various mechanical and aesthetic requirements of the ornamental articles, new alloys have been proposed, said alloys being obtained by introducing into their composition not only metal alloying elements or base elements, but also vitreous materials or plastic materials.

In more detail, the patent U.S. Pat. No. 4,476,090 describes an alloy for articles of jewelry with a low density, which is composed of a noble metal or a noble metal alloy and by glass in a percentage amount of between 1 and 70%.

One drawback of this alloy consists in the fact that it can be cast in a mould, but cannot be pressed. A further drawback consists in the fact that it has mechanical and in particular elasticity properties which are not suitable for the production of jewelry intended for ornamental purposes.

The patent U.S. Pat. No. 5,578,383 describes an alloy for articles of jewelry with a low density, which is composed of a noble metal or a noble metal alloy in a percentage amount of between 33 and 99% and a percentage amount of between 1 and 67% of a thermosetting crosslinkable polymer.

In accordance with the production techniques described in the two abovementioned patents, the end product is obtained by mixing two or more mutually reactive liquid precursors which are poured into a mould and which, as a result of their mutual reaction, are converted into a crosslinked solid which cannot be shaped by the action of heat.

In other words, the articles of the type described hitherto all have the drawback that they require a complex production process which is not practical and unsuitable for achieving a high dispersion of the metallic powder within the polymer or vitreous matrix, which is able in particular to ensure the same concentration or the same purity index at all points within the product.

Although these alloys have good stability characteristics, often the metallic powders contained in the polymer matrix do not form strong bonds and may therefore be easily released by the surface of the finished articles and therefore dispersed in the environment.

Moreover, mixing of components with such varying characteristics such as metals and polymers is not performed using methods able to ensure that optimum mechanical characteristics such as elasticity and surface micro-roughness are achieved in the finished products.

The patent U.S. Pat. No. 4,282,174 describes a technique for producing articles for the gold and silver industry which envisages producing beforehand plastics powders with very fine dimensions and combining the plastics powders with metal powders having dimensions of between ½ and 50 micron, resulting in a mixture of powders which are used for pressing at high pressures and at temperatures of between 100 and 250° C. in order to obtain the articles with the desired shape and size.

A first drawback of the production process described in this patent consists in the use of complex processes in order to obtain the pulverized plastic material.

A second drawback consists in the use of thermoplastic polymers such as polyethylene, polystyrene, polymethylmethacrylate and polyamide which have a limited resistance, namely a high hardness factor and poor elasticity.

By way of example the typical elongation values of the materials mentioned are given below:

Polyamides: 5-200%;

Polyesters: 3-5%

Polymethylmethacrylate: 1.5-10%;

Low-density polyethylene: values higher than 400%, but with 20% expansion under pulling stress, namely the polymer stretches irreversibly. Moreover, it has a melting temperature in the range of 100-115° C. and a Vicat softening temperature B/50 <40° C., resulting in this polymer having a limited consistency already at low temperatures and therefore being unsuitable for use in the production of gold and silverware articles.

Therefore the materials considered in the abovementioned patent U.S. Pat. No. 4,282,174 are not suitable for obtaining materials and articles with satisfactory mechanical important properties. It is in fact extremely important to obtain precious materials which are sufficiently resistant and elastic such that they do not disintegrate with time and, during the use in question, are also able to guarantee the economic value of the articles which they must produce.

The production method for forming the ornamental articles is performed by dry mixing the particles of the polymer component and metal component in the powder state and then by means of subsequent injection in a mould.

This production method has, in practice, proved to be unsuitable for obtaining jewelry with superior mechanical properties and in particular with a highly uniform dispersion of the precious metal in the polymer matrix.

It should be pointed out again that the technologies known hitherto, for mixing polymer compositions with precious metal powders in order to produce jewelry, have adopted dry mixing of the powders of the two materials. These technologies have been unable to achieve a satisfactory dispersion of the metal powders in the polymer matrix such as to obtain a homogenization able to produce ornamental articles which have superior mechanical properties and a purity index which constant at all points in the product mass.

### DISCLOSURE OF THE INVENTION

In this situation, the main object of the present invention is to provide a method for producing a precious material for ornamental articles which allows a homogeneous product to be obtained, having mechanical properties which are able to ensure a notable degree of comfort when the product is worn and are distinguished by a high elasticity.

Another object of the present invention is to provide a precious material for ornamental articles which is extremely homogeneous and able to ensure a highly uniform concentration of the constituents throughout the product.

A further object of the present invention is to provide a method for producing a precious material for ornamental articles which is simple to produce and operationally entirely reliable.

A further object of the present invention is to provide a precious material for ornamental articles which has a homogeneous concentration of precious metal and a high elasticity.

These and other objects are all achieved by the method for producing a precious material for ornamental articles and by the ornamental articles which can be obtained in particular by means of this method, in accordance with the accompanying claims.

### DETAILED DESCRIPTION

The technical features of the invention, in accordance with the abovementioned objects, may be clearly determined from the contents of the claims below and the advantages therefore will emerge more clearly from the detailed description which follows, provided with reference to a purely exemplary and non-limiting embodiment of the said invention.

The precious material according to the present invention may be advantageously used as an intermediate product in the jewelry industry in order to produce ornamental articles which contain percentages of precious metal which are equal to those of any precious-metal alloy, improving the mechanical properties such as, in particular, the elasticity.

Alternatively, this intermediate product may be used, not only for forming an ornamental article of jewelry but also for forming accessories or inserts for products in the clock and watch-making, spectacles, clothing, footwear and/or leatherwear sector, since these precious materials—precisely because of their elasticity and resistance—are, at suitable thicknesses, suitable for stitching.

The method envisages a step for preparation of a powder of one or more precious metals present individually, or in an alloy or in combination. This powder is formed with particles having an average size of less than 0.5 micron.

In particular the precious metals are chosen from the group containing: gold, silver, platinum or alloys thereof.

The components of a thermoplastic polymer composition are then prepared and introduced in granular form or separately by means of a first gravimetric metering device in an overall percentage by weight ranging between 30 and 95%, inside a plasticization chamber of an extruder.

The percentages of powders and polymeric composition are chosen depending on the purity index (or the percentage of precious metal) which is ultimately to be obtained in the finished product.

The chamber of the extruder is heated to a temperature of between 120-220° C. in order to melt and mix thoroughly the constituents of the thermoplastic composition.

By means of a second gravimetric metering device the metallic powder is then introduced and dispersed within the liquid thermoplastic polymer composition, in an overall percentage amount by weight ranging between 30 and 95% chosen so as to obtain the desired purity in the finished product.

The plasticization chamber (cylinder) has inside it one or more screws (or augers) composed of conveying segments and mixing segments which, at the cylinder temperature of between 120 and 220° C. and at predefined speeds of rotation, perform optimum mixing of the polymer composition in the liquid state and the amount of powder/metallic powder which obviously does not reach the melted state.

The extruder used will preferably be one of the single-screw or twin-screw high-shear type able to achieve homogeneous dispersion of the precious metal within the liquid polymer matrix.

Considering that the components of the precious metals have substantially different specific weights and that it is of fundamental importance to prevent sedimentation of the metal in order to avoid a discontinuity in the composition, as an alternative to the gravimetric metering devices, it is possible to use in accordance with a further embodiment of the present invention, also predispersed compositions of precious metal in low-melting thermoplastic polymers, compatible with the base polymer.

The liquefied mix of metallic powders in the polymer matrix undergoes extrusion, producing an intermediate product in the form of a bead (and/or thin strip).

The intermediate product obtained from the abovementioned extrusion operation is a composite (or “compound” in the technical jargon of the sector) namely a combination of several materials which are thoroughly mixed together.

Examinations carried out on various points of the bead samples produced results for the percentage precious metal content which corresponded to the purity index initially established, thus demonstrating the perfect homogeneity achieved.

Finally, the bead may be used as a base material for a step involving forming of the ornamental articles which is to be produced.

Preferably, upon leaving the extruder, the intermediate product in the form of a bead will be cut or shaped into granules having the same original purity index in the form of prismatic, spherical or cylindrical elements.

For the purposes of the present invention it is important that the mixing step which results in formation of the intermediate product should be separate from the forming step. As a result it is possible to optimize the extrusion conditions in terms of solely the mixing constraints, with the advantage of obtaining an intermediate product which is extremely homogeneous.

Advantageously, the intermediate product may be stored for subsequent use in the forming process which may therefore be performed in a location different from that for production of the intermediate product and also at a considerably later time.

The forming operation which results in the production of the ornamental articles with the desired carat value may envisage, in accordance with a preferred solution of the present invention, supplying the granular intermediate product to an injection moulding press, or an intrusion moulding press or a compression moulding press, operating at temperatures of between 120 and 220° C.

Using these conversion technologies it is possible to obtain articles which are shaped with the final form or parts which are overmoulded onto pre-formed parts.

Alternatively, the forming step may envisage supplying the intermediate granular product to an extrusion machine operating at temperatures of between 120 and 220° C., obtaining continuous shaped profiles.

By way of yet another alternative, the forming step may envisage supplying a cylinder calendering machine so as to obtain a continuous flat and/or foil-like strip which may or may not be embossed and which may undergo further processing steps in order to obtain the finished product.

The intrusion moulding press, the compression moulding press, the extrusion machine for obtaining continuous shaped profiles and the cylinder calendering machine for obtaining a continuous strip are machines which are well-known per se to a person skilled in the art and for this reason are not described in detail; however their use in the forming process, based on the intermediate product described, is entirely novel and original.

Once the product has been formed it may undergo other processing operations in order to produce the finished product, which may also be stitched onto a fabric.

The present invention also relates to a precious material for ornamental articles which can be obtained as an intermediate product of the method described above.

Similarly, the method described above may be further characterized with reference to the characteristics of the precious material indicated in the description which follows.

The precious material is obtained as explained above by starting with a suitable amount of precious metal powders present in an alloy or individually with an average size of less than 0.5 micron and by an amount of a homogeneous thermoplastic polymer composition, which amounts are mixed in an extruder.

In greater detail, the polymer composition comprises thermoplastic polymers which are obtained by means of polycondensation of one or more resins which are chosen from the group comprising: thermoplastic polyurethanes, copolysters and copolyamides.

These thermoplastic polymers may be further elastomerized with compatibilized styrene block resins and/or with other known elastomerizing agents such as nitrile and butadiene resins.

A particular polymer composition which is particularly sui table for the use according to the objects of the present invention is, by way of example, represented by a thermoplastic polyurethane obtained by means of reaction between aromatic and aliphatic isocyanates, ester base polyols, glycol polytetramethylene ester, carbonate ester with a molecular weight of between 1000 and 4000 and diol chain extenders with a molecular weight of 50 to 400; this reaction may be achieved either by means of the two-step process, which envisages supplying the liquid monomers into a reactive extruder in an operating condition such as to favour the formation of a solid intermediate product, which is subsequently re-extruded and granulated in a second non-reactive extruder, or using the prepolymer process, which envisages a first reaction inside a chemical reactor between part of the components of the formulation, resulting in a reactive liquid intermediate product (called “prepolymer”) subsequently supplied together with the remaining components of the formulation into a reactive extruder, or using “one-shot” reactive extrusion technology in which all the monomers are supplied into the reactive extruder, the operating mode of which is configured to obtain the granulated finished product.

Preferably, the thermoplastic polyurethanes comprise aliphatic isocyanates, carbonate esters of the medical type with a molecular weight of between 400 and 4000, and chain extenders with a molecular weight of between 50 and 400.

The choice of this particular type of thermoplastic polyurethane is advantageously due to the particular biostability which makes it suitable for contact with the skin of any person wearing the ornamental product.

The thermoplastic composition must have non-toxic, anti-allergic and anti-microbial properties since it has to come into contact with the human skin.

For this purpose the polymer composition or the precious material also comprise an additive with an anti-microbial action incorporated during the production method in a plasticization chamber and preferably consisting of solution of 2-octyl-2H-isothiazol-3-one and Triclosan, which is known by the tradename of “Sanitized”, and is present in percentage amount varying from 0.3 to 1% by weight.

The precious material composition also contains UV and temperature stabilizers in an amount suitable for best determining the properties thereof as regards resistance to ageing and formation of bacterial colonies. The precious material has an elongation at break value in the range of 200-800% and a hardness factor of between 50 Shore A and 70 Shore D.

By way of a non-exhaustive illustration, the following example is given.

170 g of aliphatic TPU with carbonate ester base (hardness Shore A 73) are mixed thoroughly inside the extruder with 300 g of gold having a purity index of 916 (22 carats) in a single-screw extruder at 160-180° C. and cut into granules using a knife cutter after cooling in water.

The granules thus obtained were dried in an oven at 70° C. for two hours and then processed at 160° C. in a hot-cylinder calender so as to obtain a sheet of thermoplastic precious material with a gold purity index of 586 (14 carats).

This precious material has the following physical and 5 mechanical characteristics:

hardness Shore A: 60 (ASTM D 2240)

breaking load: 8.1 Mpa (ASTM D 412)

elongation at break: 630% (ASTM D 412)

Precious metals such as gold, silver and platinum are heavy metals with a density much greater than that of the thermoplastic composition in which they are dispersed inside the precious material.

Therefore, an ornamental article also with a high carat value (precious metal percentage) will be distinguished by a volume of polymeric composition which is far greater than the volume of metallic component.

For example, considering that gold is a heavy metal with a density of 19.3 g/cm³ and that the density of an elastomer composition indicated above may vary between 1 and 1.5 g/cm³, it follows that the volumetric ratio of gold and elastomer composition is substantially biased in favour of the latter.

In other words, very roughly, an 18-carat precious material could be formed by 75 parts by weight of gold and 25 parts by weight of elastomer polymer which, in terms of volume, may be represented by 3.88 cm³ of gold and 20.83 cm³ of elastomer with a density 1.2 g/cm³.

The precious material obtained according to the method and according to the characteristic features of the invention described above allows a high dispersion of the metallic powders inside the elastomer plastic matrix, guaranteeing the purity and the mechanical performance throughout the finished product and ensuring a surprising and favourable tactile feel.

With the precious material according to the invention, it is possible to produce ornamental articles with the desired purity index (i.e. with a guarantee as to the desired percentage of precious metal) such as, for example, in the gold and silverware sector, necklaces, bracelets, rings, earrings, etc., which have a final aesthetic appearance which is distinguished by a highly appreciated tactile feel and by mechanical properties represented in particular by the hardness and the elasticity which allow extremely versatile use of the precious material.

The cost of these articles, considering the simplicity and the repetitiveness of the production process, and in particular of the forming process, is less than the cost of the conventional method for producing precious metal articles since it does not envisage the surface treatments such as polishing and diamond-machining which form part of the conventional process.

Advantageously the use of the precious material according to the present invention is not limited solely to the jewelry sector, but also extends to other sectors for example the clock and watch-making, gift article, clothing, footwear or leatherwear sector, considering the fact that these elastic and resistant precious material, at suitable thicknesses, can be stitched.

1. Method for producing a precious material for ornamental articles, which comprises:
a step for preparing at least one powder of one of more precious metals present individually, or in an alloy, or in combination;
a step for preparing at least one thermoplastic polymer composition;
a step for mixing at least one amount of said polymer composition and at least one amount of said metallic powder;
characterized in that:
said powder of said step for preparing the powder, is formed by particles having an average size of less than 0.5 micron;
said polymer composition of said step for preparing at least one thermoplastic polymer composition, is obtained from elastomeric thermoplastic polymers;
said method comprises a step for introducing, into the plasticization chamber of an extruder, said amount of said polymer composition in a percentage by weight ranging between 70 and 5% and said amount of said metallic powder in a percentage by weight ranging between 30 and 95%;
said step for mixing is a thorough mixing at a temperature of between 120 and 220° C. said amount of polymer composition in the liquid state and said amount of metallic powder by means of at least one screw provided with conveying and mixing segments;
said procedure comprises moreover:
a step for extruding the mixture thus obtained resulting in an intermediate product with a precious metal content and in the form of a bead and a step for forming the ornamental article from said intermediate product.

2. Method according to claim 1, characterized in that it comprises a step for separating said bead into granules so as to form a granular intermediate product, in particular with the granules in the form of prismatic, spherical or cylindrical elements.

3. Method according to claim 1, in which introduction of said thermoplastic composition inside said chamber is performed by means of a first gravimetric metering device.

4. Method according to claim 1, in which said amount of metallic powder is introduced by means of a gravimetric metering device inside said plasticization chamber where said thermoplastic polymer composition is in the liquid state.

5. Method according to claim 1, in which said amount of thermoplastic polymer composition is introduced into said extruder in granular form.

6. Method according to claim 2, in which said forming step envisages supplying said granular intermediate product to an injection moulding, intrusion moulding or compression moulding press operating at temperatures of between 120 and 220° C.

7. Method according to claim 2, in which said forming step envisages supplying said granular intermediate product to an extrusion machine operating at temperatures of between 120 and 220° C.

8. Method according to claim 1, in which said forming step envisages supplying a hot-cylinder calendering machine.

9. Method according to claim 8, in which said forming step produces an ornamental article of jewelry or an accessory or an insert for products in the clock and watch-making, spectacles, clothing, footwear and/or leatherwear sector, in view of the fact these precious materials, precisely because they are elastic and resistant, at suitable thicknesses, can be stitched.

10. Precious material for ornamental articles that comprises a metallic composition formed by one or more precious metals present in an alloy, in combination or individually and a thermoplastic polymer composition binding said precious metals within the precious material, characterized in that:
said precious metals have an average size of less than 0.5 micron and with an overall content by weight in the range of between 30 and 95%;
said thermoplastic polymer composition has an overall content by weight in the range of between 70 and 5%;
said metallic and polymer compositions have in an alloy a homogeneity resulting from mixing in an extruder;
said polymer composition comprising thermoplastic elastomeric polymers obtained from polycondensation and chosen from the group comprising: thermoplastic polyurethanes, copolyesters and copolyamides;
said alloy has an elongation at break in the range of 200-800% and hardness of between 50 Shore A and 70 Shore D.

11. Precious material for ornamental articles according to claim 10, in which said thermoplastic polyurethanes comprise aliphatic isocyanates, carbonate ester of the medical type with a molecular weight of between 400 and 4000, and chain extenders with a molecular weight of between 50 and 400, in suitable and selected molar ratios.

12. Precious material for ornamental articles according to claim 11, in which said thermoplastic polymers (polyurethanes, copolyesters and copolyamides) may be further elastomerized with compatibilized styrene block resins and/or with other known elastomerizing agents such as nitrile and butadiene resins.

13. Precious material for ornamental articles according to claim 10, in which said precious metals are chosen from within the group containing: gold, silver, platinum or alloys thereof.

14. Precious material for ornamental articles according to claim 10, characterized in that it comprises an additive with an anti-microbial action, preferably consisting of a solution of 2-octyl-2H-isothiazol-3-one and Triclosan, which is known by the tradename of Sanitized™, and is present in a percentage amount varying from 0.3 to 1% by weight.
